(19) United States
(12) Patent Application Publication
(10) Pub. No.: US 2003/0027199 A1
(43) Pub. Date: Feb. 6, 2003

(54) Measurement chip for biosensor

(76) Inventor: Masayoshi Kojima, Saitama (JP)

Correspondence Address: Jules E. Goldberg, Esq., Reed Smith LLP, 375 Park Avenue, New Yok, NY, 10152, US

(21) Appl. No.: 10/236,398

(22) Filed: Sep. 6, 2002

Related U.S. Application Data
(62) Division of application No. 09/895,512, filed on Jun. 28, 2001.

(30) Foreign Application Priority Data
Jun. 29, 2000 (JP) 2000-196371

(51) Int. Cl. C12Q 1/68; C12M 1/34; B05D 3/00

(52) U.S. Cl. 435/6; 435/287.2; 427/2.11

(57) ABSTRACT

The object of the present invention is to provide a method for easily immobilizing a physiologically active substance to a metal surface wherein the processes are simple and highly safe. According to the present invention, there is provided a measurement chip for a biosensor comprising a metal surface or a metal membrane which was treated with a compound represented by the following formula I:

X—A—Y Formula I

wherein X represents a heterocyclic group comprising a Z—Z bond in a ring thereof wherein Z represents S, Se or P;

A represents a divalent linking group selected from an aliphatic group, an aromatic group, a heterocyclic group or a combination thereof; and

Y represents a functional group capable of covalently binding to a physiologically active substance.

### TECHNICAL FIELD OF THE INVENTION

The present invention relates to a measurement chip for a biosensor having a metal surface or a metal membrane which was treated with a linker compound capable of binding to a physiologically active substance, and a method for immobilizing a physiologically active substance to a metal surface or a metal membrane using the above linker compound.

### BACKGROUND ART

Presently, a large number of measurements using immune response are carried out in clinical tests etc. However, since conventional methods require complicated operations or labeling substances, an immunosensor is used which employs surface plasmon resonance (SPR) capable of detecting change of ligand with high sensitivity, without requiring a labeling substance.

In a measurement chip commonly used for a measurement device (a surface plasmon resonance biosensor) employing such a surface plasmon resonance, porous materials are formed on a metal membrane coated on a glass substrate, and a physiologically active substance such as an enzyme and an antibody is deposited or immobilized on the surface of or inside of these porous materials. Examples of these porous materials include a textile fabric, a knitting fabric and a nonwoven fabric which are made of synthetic fibers, natural fibers, inorganic fibers etc., and porous inorganic or organic materials (see Japanese Patent Application Laid-Open (kokai) No. 3-164195). Moreover, for a commercial product (BIAcore 2,000, Pharmacia Biosensor), carboxymethyl dextran is used as a porous material.

Nevertheless, since physiologically active substances which substantially and efficiently interact with a subject of measurement are only those which exist on the surface of a porous material, a physiologically active substance deposited or immobilized inside the porous material does not function effectively, resulting in reduction of sensitivity.

As a method for immobilizing a physiologically active substance on a metal membrane, LB (Langmuir-Blodgett) method may be used (see Japanese Patent Application Laid-Open (kokai) No. 5-288672), but this method has a problem in that the binding between an LB membrane and a metal membrane is so weak that the LB membrane falls off together with the physiologically active substance.

Since various types of compounds, which are directed to bind to a metal surface, comprise S, P, Se etc. (Japanese Patent Application Laid-Open (kohyo) No. 4-501605), attention must be paid to odor, toxicity etc., when these compounds are handled.

### SUMMARY OF THE INVENTION

The object of the present invention is to solve the above-stated problems of the prior art. That is to say, the object of the present invention is to provide a method for easily immobilizing a physiologically active substance to a metal surface wherein the processes are simple and highly safe.

As a result of thorough studies by the present inventors directed toward the above object, we have found that a metal surface having a functional group capable of immobilizing a physiologically active substance can be produced by treating the surface of a metal membrane with a compound of formula I defined in the present specification, thereby completing the present invention.

Thus, according to the present invention, there is provided a measurement chip for a biosensor comprising a metal surface or a metal membrane which was treated with a compound represented by the following formula I:

X—A—Y Formula I

wherein X represents a heterocyclic group comprising a Z—Z bond in a ring thereof wherein Z represents S, Se or P;

A represents a divalent linking group selected from an aliphatic group, an aromatic group, a heterocyclic group or a combination thereof and

Y represents a functional group capable of covalently binding to a physiologically active substance.

In the measurement chip for a biosensor of the present invention, X in formula I is preferably a heterocyclic group having the following structure:

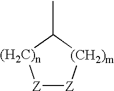

wherein

Z represents S, Se or P; and

m and n independently represent an integer of 0 or more.

In the measurement chip for a biosensor of the present invention, X in formula I preferably represents a 1,2-dithiolane group.

In the measurement chip for a biosensor of the present invention, Y in formula I preferably represents —OH, —COOH, —NH₂, —CHO, —NHNH₂, —NCS, an epoxy group or a vinyl group.

In the measurement chip for a biosensor of the present invention, the compound represented by formula I is lipoic acid having the following structure:

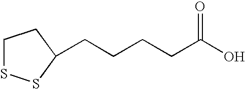

In one embodiment of the measurement chip for a biosensor of the present invention, a physiologically active substance is bound to a compound of formula I. The physiologically active substance is preferably an immune protein, enzyme, microorganism, nucleic acid, low molecular weight organic compound, non-immune protein, immunoglobulin binding-protein, sugar-binding protein, sugar chain recognizing sugar, fatty acid or fatty acid ester, or polypeptide or oligopeptide capable of binding to a ligand.

According to another aspect of the present invention, there is provided a biosensor comprising the measurement chip for a biosensor of the present invention stated above.

According to still another aspect of the present invention, there is provided a method for detecting and/or measuring a substance which interacts with a physiologically active substance, using the measurement chip for a biosensor or the biosensor of the present invention as mentioned above, wherein the above physiologically active substance is immobilized to the above measurement chip for a biosensor.

According to still another aspect of the present invention, there is provided a method for immobilizing a physiologically active substance to a metal surface or a metal membrane, which comprises the steps of treating the above metal surface or metal membrane with a compound represented by the following formula I:

X—A—Y Formula I

wherein X represents a heterocyclic group comprising a Z—Z bond in a ring thereof wherein Z represents S, Se or P;

A represents a divalent linking group selected from an aliphatic group, an aromatic group, a heterocyclic group or a combination thereof, and

Y represents a functional group capable of covalently binding to the physiologically active substance, and

binding the above physiologically active substance to a compound represented by formula I directly or via a crosslinking compound.

In the method for immobilizing a physiologically active substance according to the present invention, X in formula I preferably represents a heterocyclic group having the following structure:

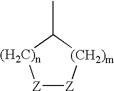

wherein

Z represents S, Se or P; and

m and n independently represent an integer of 0 or more.

In the method for immobilizing a physiologically active substance according to the present invention, X in formula I preferably represents a 1,2-dithiolane group.

In the method for immobilizing a physiologically active substance according to the present invention, Y in formula I preferably represents —OH, —COOH, —NH₂, —CHO, —NHNH₂, —NCS, an epoxy group or a vinyl group.

In the method for immobilizing a physiologically active substance according to the present invention, the compound represented by formula I is preferably lipoic acid having the following structure:

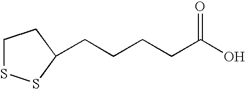

### DETAILED DESCRIPTION OF THE INVENTION

The embodiments and methods for carrying out the present invention are described in detail below.

The measurement chip for a biosensor of the present invention is characterized in that it comprises a metal surface or a metal membrane which was treated with a compound represented by the following formula I:

X—A—Y Formula I

wherein X represents a heterocyclic group comprising a Z—Z bond in a ring thereof wherein Z represents S, Se or P;

A represents a divalent linking group selected from an aliphatic group, an aromatic group, a heterocyclic group or a combination thereof; and

Y represents a functional group capable of covalently binding to a physiologically active substance.

The measurement chip for a biosensor of the present invention can be used, for example, as a measurement chip for surface plasmon resonance biosensor etc, which is characterized in that it is equipped with a metal membrane located on a transparent substrate.

A measurement chip for surface plasmon resonance biosensor is herein used to mean a chip used for a surface plasmon resonance biosensor, which is a member comprising a portion for transmitting and reflecting light emitted from the above sensor and another portion for immobilizing a physiologically active substance. The member may be fixed to the body of the above sensor, or may be removable.

Surface plasmon resonance is a phenomenon which occurs as a result of that the intensity of monochromatic light reflected from a boundary between a optically transparent substance such as glass and a metal thin layer depends on the refractive index of a sample located at the irradiation side of the metal. Therefore, a sample can be analyzed by measuring the intensity of monochromatic light reflected.

The measurement chip for a biosensor of the present invention is produced by treating a metal surface or a metal membrane with a compound of formula I defined in the present specification.

A metal membrane is preferably located on a substrate. The term “located on a substrate” is used herein to comprise the case where a metal membrane is located to directly contact with a substrate, as well as the case where a metal membrane does not directly contact with a substrate but contacts via another layer.

In the case where a metal membrane is located on a substrate, the measurement chip for a biosensor of the present invention has a substrate, a metal membrane formed on the substrate, and a linker layer formed on the metal membrane (comprising a compound of formula I).

With regard to a substrate which can be used in the present invention, for example, for a surface plasmon resonance biosensor, any substrate may be used, as long as it is applicable in an immobilization method. Generally, there can be used materials transparent to a laser beam, such as glass, polyethylene terephthalate and polycarbonate. Such substrates are preferably made of a material which is not anisotropic to polarization and has an excellent processing property. The thickness of substrate is not particularly limited, but normally it is about 0.1 to 20 mm.

With regard to a metal membrane for the measurement chip for a biosensor of the present invention, in the case of a surface plasmon resonance biosensor, any metal membrane can be applied, as long as it can bring about surface plasmon resonance. Examples of metal which can be used for this metal membrane include gold, silver, copper, aluminum, platinum etc., and these can be used alone or in combination. Furthermore, taking the adherence of the metal to the above substrate into account, an interstitial layer consisting of chromium or the like may be provided between the substrate and the layer consisting of gold, silver etc.

The thickness of the metal membrane is not particularly limited, but for example, for a surface plasmon resonance biosensor, it is preferably 100 to 2,000 angstrom, and is particularly preferably 200 to 600 angstrom. If the thickness is more than 3,000 angstrom, it becomes impossible to sufficiently detect the surface plasmon phenomenon of medium. Moreover, in the case where an interstitial layer such as chromium is provided, the thickness of the layer is preferably 5 to 50 angstrom.

The formation of a metal membrane may be carried out according to standard techniques such as sputtering, evaporation, ion plating, electroplating and electroless plating.

In the present invention, a compound represented by the following formula I is used:

X—A—Y Formula I

wherein

X represents a heterocyclic group comprising a Z—Z bond in a ring thereof wherein Z represents S, Se or P;

A represents a divalent linking group selected from an aliphatic group, an aromatic group, a heterocyclic group or a combination thereof, and

Y represents a functional group capable of covalently binding to a physiologically active substance.

In formula I, X is a heterocyclic group comprising —S—S—, —Se—Se—, or —P—P— in a ring thereof. X is preferably a heterocyclic group having the following structure:

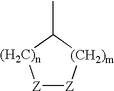

wherein

Z represents S, Se or P; and

m and n independently represent an integer of 0 or more.

The upper limits of m and n are not particularly limited, but from the viewpoint of convenience of chemical synthesis, the total number of m+n is preferably 0 to 10, and more preferably 0 to 7, further preferably 0 to 5, and further more preferably 2 or 3.

X is particularly preferably a 1,2-dithiolane group.

In formula I, A represents a divalent linking group selected from an aliphatic group, an aromatic group, a heterocyclic group or a combination thereof.

Examples of an aliphatic group include alkylene, alkenylene, alkynylene etc., and the form of a chain may be a linear chain, a branched chain, a cyclic chain or a combination thereof. As an aliphatic group, alkylene is particularly preferable, and linear alkylene is most preferable. The length of an aliphatic group is not particularly limited, and for example, 1 to 20 carbon atoms, more preferably 1 to 10 carbon atoms, and particularly preferably 2 to 10 carbon atoms.

Examples of an aromatic group include arylene group etc., and more specifically, phenylene group, naphthylene group etc.

Examples of a heterocycle include a 5-or 7-membered saturated or unsaturated monocycle or condensed cycle having one or more hetero atoms selected from nitrogen, oxygen or sulfur, and specific examples include pyridine, quinoline, isoquinoline, pyrimidine, pyrazine, pyridadine, phthalazine, triazine, furan, thiophene, pyrrole, oxazole, benzoxazole, thiazole, benzothiazole, imidazole, benzimidazole, thiadiazole, triazole, etc. The term heterocyclic group means a divalent group derived from the aforementioned heterocycles.

A divalent linking group represented by A may also be constructed from the combination of an aliphatic, aromatic or heterocyclic group as stated above.

In formula I, Y represents a functional group capable of covalently binding to a physiologically active substance. Examples thereof include —OH, —COOH, —NH₂, —CHO, —NHNH₂, —NCS, an epoxy group or a vinyl group, and —COOH is particularly preferable.

Among compounds represented by formula I, lipoic acid, which is easily dissolved in basic water, alcohol etc., being easy-to-handle, is preferably used. Lipoic acid (thioctic acid; Wako Pure Chemical Industries, Ltd.) comprises a carboxyl group and is capable of binding its dithiirane portion to a metal surface, thereby the carboxyl group can be effectively used to immobilize a physiologically active substance.

As an example of the use of a lipoic acid derivative to reform interface conditions, adherence of resin to a dental metal has been reported (Yoshinori Kadoma, Shika-Zairyo (Dental Materials & Appliances), vol. 16 (2), 114-121 (1997)).

Compounds of formula I can be synthesized by a common organic chemical synthesis known to a person skilled in the art. Specifically, the compounds can be synthesized according to a synthetic method described in e.g. Ralph G. Nuzzo and David L. Allara, “Adsorption of Bifunctional Organic Disulfides on Gold Surfaces”, J. Amer Chem. Soc, 105, 4481-4483 (1983).

Examples of a method for treating a metal surface or a metal membrane with a compound of formula I include a method of dipping a metal membrane etc. into a solution containing the above compound for a certain period of time (dip method), a method of using Spin Coater (spin coating), a method of using a gravure printing press (photogravure) etc.

The compound (a linker compound) of formula I used in the present invention has the following advantages:

(1) Since the linker compound allows a physiologically active substance to be immobilized at a position extremely close to a metal membrane, measurement sensitivity can be highly improved as compared with the conventional immobilization methods.

(2) Surface treatment is easy and a large amount of surface treatment can be carried out at a time.

(3) By the choice of Y which is a functional group capable of covalently binding to a physiologically active substance, it becomes possible to perform a chemical modification such as a surface reforming, the introduction of other functional groups etc.

In respect of the measurement chip for a biosensor, a physiologically active substance is immobilized to a metal surface which was treated with the compound (a linker compound) of formula I directly or via a crosslinking reagent (e.g. a water-soluble multivalent reagent etc.)

Examples of the crosslinking reagent include glutaraldehyde, periodic acid, N-succinimydyl-2-maleimide acetic acid, N-succinimydyl-4-maleimide butyric acid, N-succinimydyl-6-maleimide hexanoic acid, N-succinimydyl-4-maleimidemethylcyclohexane-1-carboxylic acid, N-sulfosuccinimydyl-4-maleimidemethylcyclohexane-1-carboxylic acid, N-succinimydyl-4-maleimidemethyl benzoic acid, N-succinimydyl-3-maleimide benzoic acid, N-sulfosuccinimydyl-3-maleimide benzoic acid, N-succinimydyl-4-maleimidephenyl-4-butyric acid, N-sulfosuccinimydyl-4-maleimidephenyl-4-butyric acid, NN′-oxydimethylene-dimaleimide, NN′-O-phenylene-dimaleimide, N,N′-m-phenylene-dimaleimide, N,N′-p-phenylene-dimaleimide, N,N′-hexamethylene-dimaleimide, N-succinimydylmaleimide carboxylic acid, N-succinimydyl-S-acetylmercaptoacetic acid, N-succinimydyl-3-(2-pyridyldithio)propionate, S-acetylmercapto succinic anhydride, methyl-3-(4′-dithiopyridyl)propionimidate, methyl-4-mercaptobutylimidate, methyl-3-mercaptopropionimidate, iminothiolene, O-carboxymethyl-hydroxylamine, azodiphenylbismaleimide, bis(sulfosuccinimydyl)suberate, 4,4′-diisothio-cyano-2,2′-disulfonic acid stilbene, 4,4′-difluoro-3,3′-dinitrodiphenylsulfone, 1,5-difluoro-2,4-dinitrobenzene, p-phenylenediisothiocyanate, dimethyladipimidate, dimethylpimelimidate, dimethylsperimidate, p-azidephenacylbromide, p-azidephenylglyoxal, N-hydroxysuccinimydyl-4-azidebenzoate, 4-fluoro-3-nitrophenylazide, methyl-4-azidebenzoimidate, N-5-azide-2-nitrobenzoyloxysuccimide, N-succinimydyl-6-(4′-azide-2′-nitrophenylamino)hexanoate, 1,4-benzochinone, N-succinimydyl-3-(2′-pyridyldithio)propionate, sodium N-(4-maleimidebutyloxy)sulfosuccinimide salt, sodium N-(6-maleimidecaproyloxy) sulfosuccinimide salt, sodium N-(8-maleimidecaproyloxy)sulfosuccinimide salt, sodium N-(11-maleimideundecanonyloxy)sulfosuccinimide salt, N-[2-(1-piperazinyl) ethyl]maleimide dihydrochloride, bisdiazobenzidine, hexamethylene disocyanate, toluene disocyanate, hexamethylene disothiocyanate, N,N′-ethylene bismaleinimide, N,N′-polymethylene bisiodoacetamide, sodium 2,4-dinitrobenzenesulfonate salt, a carbodiimide derivative wherein a diazo compound or a condensation reagent is represented by RN═C═NR (or R′), N-hydroxysuccimide, tri-n-butylamine, butylchloroformate, isobutyl isocyanide and the like.

A physiologically active substance which is immobilized to the measurement chip for a biosensor of the present invention is not particularly limited, as long as it interacts with a measurement subject, and examples thereof include immune protein, enzyme, microorganism, nucleic acid, low molecular organic compound, non-immune protein, immunoglobulin binding-protein, sugar-binding protein, sugar chain recognizing sugar, fatty acid or fatty acid ester, and poypeptide or oligopeptide capable of binding to a ligand.

Examples of an immune protein include an antibody, a hapten and the like, the antigen of which is a measurement subject. Examples of an antibody include various immunoglobulins such as IgG, IgM, IgA, IgE and IgD. Specifically, when a measurement subject is human serum albumin, an anti-human serum albumin antibody can be used as an antibody. When a pesticide, an insecticide, methicillia resistant Staphylococcus aureus, an antibiotic, narcotic, cocaine, heroin or crack is used as an antigen, there can be used, for example, an anti-atrazine antibody, an anti-kanamycin antibody, an anti-metamphetamine antibody or antibodies against 0 antigens 26, 86, 55, 111, 157 etc. in enteropathogenic Escherichia coli.

An enzyme to be used herein is not particularly limited, as long as it shows activity against a measurement subject or a substance metabolized from the measurement subject. Various enzymes such as oxidoreductase, hydrolase, isomerase, lyase, synthetase can be used. Specifically, when a measurement subject is glucose, glucose oxidase can be used, and when a measurement subject is cholesterol, cholesterol oxidase can be used. Further, when a pesticide, an insecticide, methicillia resistant Staphylococcus aureus, an antibiotic, narcotic, cocaine, heroin or crack is used as a measurement subject, there can be used enzymes such as acetylcholin esterase, catecholamine esterase, noradrenaline esterase and dopamine esterase, which specifically react with a substance metabolized from such a measurement subject.

With regard to a microorganism, there are no particular limits, and various microorganisms such as Escherichia coli can be used.

With regard to nucleic acid, one which complementarily hybridizes to a nucleic acid of the measurement subject can be used. As a nucleic acid, both DNA (including cDNA) and RNA can be used. The types of DNA are not particularly limited, and any one of naturally occurring DNA, recombinant DNA prepared by gene recombinant techniques and chemically synthesized DNA may be used.

As a low molecular organic compound, any compounds synthesized by a common organic chemical synthetic method can be used. It is preferred to use a compound having a functional group capable of binding to the linker compound of formula I used in the present invention directly or via a crosslinking compound.

A nonimmune protein to be used herein is not particularly limited, and avidin (streptoavidin), biotin, a receptor etc. can be used.

Examples of an immunoglobulin binding-protein to be used herein include protein A, protein G, a rheumatoid factor (RF) etc.

As a sugar-binding protein, there can be used lectin and the like.

Examples of fatty acid or fatty acid ester include stearic acid, arachidic acid, behenic acid, ethyl stearate, ethyl arachidate, ethyl behenate etc.

When a physiologically active substance is a protein such as an antibody or enzyme, or nucleic acid, the immobilization of the substance can be carried out by using an amino group, a thiol group etc. of the physiolgically active substance and allowing such a group to covalently bind to a functional group located on a metal surface. For example, the immobilization of a physiologically active substance is carried out by treating the surface of a metal membrane with lipoic acid, allowing it to be actively esterified with N-hydroxysuccinimide and WSC, and contacting a certain amount of physiologically active substance with the surface for a given period of time (in a given amount). Furthermore, a common method for immobilizing physiolgically active substance based on the avidin-biotin system where avidin or biotin is immobilized is also easily constructed, but immobilization methods are not limited thereto.

Since the strong immobilization of a physiologically active substance via the above linker allows the substance to be still immobilized even after washing, the present method has an advantage in that it can be repeatedly used for measurement.

The present invention is further described in the following examples. The examples are provided for illustrative purposes only, and are not intended to limit the scope of the invention.

### EXAMPLES

### Example 1

### Binding of an Anti-CRP Antibody to a Gold Vapor-Deposited Surface and Detection Thereof

A linker was bound to a gold vapor-deposited glass surface by the process described below, and then an anti-CRP antibody covalently bound to this linker was detected by a reaction between an anti-IgG-POD antibody and ABTS. Lipoic acid used in examples was purchased from Wako Pure Chemical Industries, Ltd. A circularly perforated mask with each hole having a diameter of 5 mm was provided onto the gold vapor-deposited surface.

(1) Process

Two sheets of 1.5 cm×1.5 cm cover glass, which was gold vapor-deposited to the thickness of about 300 angstrom, were dipped into each of 1.0% by weight lipoic acid aqueous solution (in 0.1M NaHCO₃ solution) and 1.0% by weight lipoic acid solution in ethanol, followed by surface treating at 37° C. for 4 hours. Moreover, two other gold vapor-deposited cover glasses were similarly dipped into 200 mM mercaptoacetic acid-ethanol solution, followed by surface treating at 40° C. for 3 hours (handled in a draft). After twice washing the gold vapor-deposited surface with pure water and PBS, a mask perforated with 5 mm-diameter holes was contacted with the gold vapor-deposited surface, then an antibody-binding area was defined as follows. A water-soluble carbodiimide (EDC: N-ethyl-N′-(dimethylaminopropyl)carbodiimide) was dissolved in PBS (pH 6.0) to a concentration of 4 mg/ml, and then 100 μl of the mixture was poured onto the gold surface. After leaving to stand at 37° C. for 2 hours, the surface was washed twice with pure water and PBS.

100 μl of PBS (pH 6.4) solution containing an anti-CRP antibody (1.0 μg/ml) was added onto each surface, followed by leaving at 4° C. overnight.

After twice washing each surface with pure water and PBS, 100 μl of 3% BSA was added thereto, followed by blocking at 37° C. for 2 hours. After twice washing each surface with pure water and PBS, 100 μl of PBS solution containing an anti-IgG-POD antibody (1.0 μg/ml, pH 7.4) was added onto each surface, followed by reaction at 37° C. for 2 hours. After twice washing each surface with pure water and PBS, 50 μl of ABTS solution was added onto each surface, followed by reaction at room temperature for 15 minutes. 40 μl of the colored solution was collected, and 60 μl of pure water was added thereto, followed by measuring absorbance at 415 nm with a spectrophotometer.

(2) Results

The obtained results are shown in Table 1.

TABLE 1

| | |
|---|---|
| Treating solution | ABTS coloring (Abs. 415 nm) |

| | | |
|---|---|---|
| Lipoic acid solution | 0.483 | 0.537 |
| Lipoic acid ethanol solution | 0.557 | 0.509 |
| mercaptoacetic acid | 0.491 | 0.452 |

From the results of Table 1, it was shown that a method for immobilizing a physiologically active substance to a metal surface using lipoic acid is effective equivalently or greater as compared with the general methods using thiol carboxylic acid.

### EFFECT OF THE INVENTION

The measurement chip for a biosensor of the present invention can easily be produced. With this measurement chip, it becomes possible to measure a subject substance of measurement with excellent sensitivity, even if the amount of the immobilized physiologically active substance is small.

What is claimed is:

1. A measurement chip for a biosensor comprising a metal surface or a metal membrane which was treated with a compound represented by the following formula I:
X—A—Y Formula I
wherein X represents a heterocyclic group comprising a Z—Z bond in a ring thereof wherein Z represents S, Se or P;
A represents a divalent linking group selected from an aliphatic group, an aromatic group, a heterocyclic group or a combination thereof, and
Y represents a functional group capable of covalently binding to a physiologically active substance.

2. The measurement chip for a biosensor according to claim 1, wherein, in formula I, X is a heterocyclic group having the following structure: 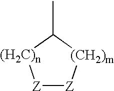
wherein
Z represents S, Se or P; and
m and n independently represent an integer of 0 or more.

3. The measurement chip for a biosensor according to claim 1, wherein, in formula I, X represents a 1,2-dithiolane group.

4. The measurement chip for a biosensor according to claim 1, wherein, in formula I, Y represents —OH, —COOH, —NH₂, —CHO, —NHNH₂, —NCS, an epoxy group or a vinyl group.

5. The measurement chip for a biosensor according to claim 1, wherein, said compound represented by formula I is lipoic acid having the following structure:

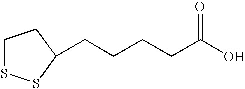

6. The measurement chip for a biosensor according to claim 1, wherein, a physiologically active substance is bound to a compound represented by formula I.

7. The measurement chip for a biosensor according to claim 6, wherein said physiologically active substance is an immune protein, enzyme, microorganism, nucleic acid, low molecular organic compound, non-immune protein, immunoglobulin binding-protein, sugar-binding protein, sugar chain recognizing sugar, fatty acid or fatty acid ester, or polypeptide or oligopeptide capable of binding to a ligand.

8. A biosensor comprising the measurement chip for a biosensor according to claim 1.

9. A method for detecting and/or measuring a substance which interacts with a physiologically active substance, using the measurement chip for a biosensor according to claim 1 or the biosensor according to claim 8, wherein said physiologically active substance is immobilized to said measurement chip for a biosensor.

10. A method for immobilizing a physiologically active substance to a metal surface or a metal membrane, which comprises the steps of treating said metal surface or said metal membrane with a compound represented by the following formula I:
X—A—Y Formula I
wherein X represents a heterocyclic group comprising a Z—Z bond in a ring thereof wherein Z represents S, Se or P;
A represents a divalent linking group selected from an aliphatic group, an aromatic group, a heterocyclic group or a combination thereof; and
Y represents a functional group capable of covalently binding to the physiologically active substance, and
binding said physiologically active substance to said compound of said formula I directly or via a crosslinking compound.

11. The method for immobilizing a physiologically active substance according to claim 10, wherein, in formula I, X represents a heterocyclic group having the following structure: 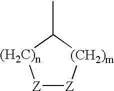
wherein
Z represents S, Se or P; and
m and n independently represent an integer of 0 or more.

12. The method for immobilizing a physiologically active substance according to claim 10, wherein, in formula I, X represents a 1,2-dithiolane group.

13. The method for immobilizing a physiologically active substance according to claim 10, wherein, in formula I, Y represents —OH, —COOH, —NH₂, —CHO, —NHNH₂, —NCS, an epoxy group or a vinyl group.

14. The method for immobilizing a physiologically active substance according to claim 10, wherein, said compound represented by formula I is lipoic acid having the following structure: [chemical structure]
